(19) United States
(12) Patent Application Publication
(10) Pub. No.: US 2019/0057779 A1
(43) Pub. Date: Feb. 21, 2019

(54) OPTIMIZATION OF OPERATION OF A MEDICAL SYSTEM

(71) Applicant: Siemens Healthcare GmbH, Erlangen (DE)

(72) Inventors: Nils Pickert, Erlangen (DE); Markus Weingarten, Bamberg (DE)

(21) Appl. No.: 16/103,910

(22) Filed: Aug. 14, 2018

(30) Foreign Application Priority Data
Aug. 15, 2017 (EP) 17186237.8

(51) Int. Cl. G16H 70/20; G16H 40/63; G16H 40/20

(52) U.S. Cl. CPC G16H 70/20; G06K 7/10297; G16H 40/20; G16H 40/63

(57) ABSTRACT

Handling of a medical system is improved. A method for operating a medical system includes acquiring parameter values that describe a current situation of the medical system finds. A situation identifier is determined for the current situation from the acquired parameter values. The parameter values are checked by corresponding values in a database, from which the corresponding values are obtained based on the determined situation identifier. A notification is then output if at least one of the parameter values for the current situation does not match one of the respective corresponding values in the database, or one of the parameter values is automatically set in accordance with the corresponding value from the database.

This application claims the benefit of EP17186237.8, filed on Aug. 15, 2017, which is hereby incorporated by reference in its entirety.

### BACKGROUND

The present embodiments relate to a method for operating a medical system and a corresponding medical system.

A medical system involves a plurality of objects that are involved in a medical procedure or at least are provided for involvement therein. A heart valve operation is, for example, one such medical procedure. For example, a surgeon, an anesthetist, and a number of assistants are the people involved in such a heart valve operation, while the items of equipment needed for such a medical procedure include, for example, an operating table, a C-arm X-ray machine, an anesthesia cart, a monitor structure, an instrument table, an ultrasound scanner, and the like. The devices may optionally also be interconnected mechanically or for data transfer. All these devices and people are in the present case denoted objects constituting part of the medical system.

Many medical procedures are always carried out by identically qualified people. A team will use the same devices for medical procedures of the same type. The same also applies to the accessories used, such as, for example, applications, workflow processes, data processing systems, consumables, and the like for the respective medical procedure.

The overall range of objects involved in a medical procedure is generally very extensive. It is therefore not unusual for one object or another needed for the medical procedure to be forgotten or to be unavailable for other reasons. This results in the simplest case merely in delays, but may also put the people involved and, in particular, the patient at risk.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method for operating a medical system with which a medical procedure may be carried out more safely or in a more goal-oriented manner and a corresponding medical system are provided.

A method for operating a medical system includes acquiring parameter values that describe a current situation in which the medical system finds itself. The medical system is, for example, composed of a plurality of objects that interact with one another to enable performance of a corresponding medical procedure. These objects may each be characterized by one or more parameter values. Such a parameter value may be a specific ID tag that is applied to the respective object. If the parameter value (e.g., the ID tag of the object) is acquired, it may be concluded that the object is present for the medical procedure. Overall, a plurality of parameter values are acquired, each of which, for example, denotes an object. Other parameters, such as, for example, temperature, brightness, atmospheric humidity, and the like or corresponding parameter values may, however, also be acquired. All of these parameter values characterize a current situation in which the medical system finds itself. In other words, the parameter values describe the status of the medical system and optionally the surroundings thereof.

A situation identifier for the current situation is then determined from the acquired parameter values. A situation identifier is, for example, assigned, based on the entirety of acquired parameter values, in accordance with a classification. Such a situation identifier may, for example, be the term “heart valve operation” for the corresponding medical procedure. A conclusion is thus automatically drawn from the individual parameter values as to the situation in which the medical system finds itself. For example, it is automatically recognized from the number and qualifications of the acquired personnel and the provided items of equipment that a heart valve operation is to be carried out. The medical system therefore itself recognizes this situation.

In a further act, the parameter values are checked using corresponding values in a database, from which the corresponding values are obtained based on the determined situation identifier. Such a database stores, for example, in one or more data records, parameter values assigned to respective situation identifiers. The database may, for example, accordingly store parameter values specifically for a heart valve operation. It is thus possible to obtain from the database those people, devices, consumables, ambient conditions, and so on that are, for example, used as standard in a corresponding medical procedure. The database may, however, also include parameter values for very specific situations (e.g., if Dr. Lopez is carrying out the heart valve operation). A parameter set for Dr. Maier may likewise be stored in the database.

In a first alternative, a notification is now output if at least one of the parameter values for the current situation does not match one of the respective corresponding values in the database. If it is thus, for example, recognized that the currently present monitor system with two monitors does not correspond to the monitor system with four monitors stored in the database, a corresponding error message or a corresponding notification may be output. In the present example, the acquired parameter value denotes the 2-monitor system, while the corresponding value in the database requires a 4-monitor system for the determined current situation or situation identifier. A corresponding notification is therefore output acoustically or visually by the medical system, such that the monitor system may, if necessary, be replaced.

Similarly, the medical system may also output a notification if, for example, an ultrasound scanner is not currently present, whereas according to the database, the ultrasound scanner ought to be present as standard in a medical procedure to which the determined situation identifier is assigned. Once the notification has been output, the missing ultrasound scanner may be added to make up the medical system appropriately.

Alternatively to outputting a notification, the method according to one or more of the present embodiments may automatically set one of the parameter values in accordance with the corresponding value from the database. Once a current situation has been acquired, one or more specific parameter values of the medical system or of the surroundings of the medical system may automatically be set in accordance with the corresponding value in the database. Thus, if, for example, a situation corresponding to the situation identifier “gallstone operation” has been recognized based on the parameter values, the ultrasound scanner is automatically set to the values for this situation that are stored in the database. Advantageously, the method according to one or more of the present embodiments thus offers the option of automatically recognizing deviations from standard situations or setting corresponding standard values. The result is a reduction in potential hazard or an increase in user convenience during medical procedures.

The parameter values may include information about which electronic objects are combined to form the medical system. In this example, at least the electronic devices and apparatuses that are part of the medical system are recognized. Such electronic objects may, for example, be registered via wireless communication links. Such electronic objects are, for example, a C-arm X-ray machine, an electronically adjustable operating table, an anesthesia system, and the like.

Specifically, it may be provided that the notification is output if, for the determined situation identifier, the corresponding values in the database reveal that at least one object is missing from the objects combined to form the medical system. The absence of an object from the medical system is thus checked, where the database serves as reference. In this manner, it may reliably be provided that a device or an apparatus that is of importance to a specific medical procedure is not missing.

A further development provides that one of the objects is a device, a tool, a data network, or a person. For example, it is identified in a current situation corresponding to a preparatory phase for an operation that the ultrasound scanner is not in the room. The ultrasound scanner may subsequently be obtained in response to a corresponding notification. A corresponding notification may also be output if the ultrasound scanner is set with parameters other than those required by the database. Alternatively, the ultrasound scanner may also be automatically set in accordance with the determined situation. The like also applies not only to tools, such as, for example, knives, saws, or hammers, but also to consumables such as, for example, screws, plates, and the like. A corresponding notification is output if the tools and/or the consumables are missing from the recognized situation. Access to a data network (e.g., hospital system) may also be checked or set. The like applies to people who should be acquired or should be present as standard in the current situation. For example, a third surgical nurse may always be present in a specified situation. Absence may also be correspondingly indicated here.

The method according to one or more of the present embodiments further provides that each of the objects has an RFID tag, and acquisition of the parameter values involves reading all the RFID tags of the objects of the medical system. RFID tags may be read wirelessly. RFID tags may contain a certain amount of information that is of significance to the medical system. For example, RFID tags may contain or transfer those parameter values necessary for checking with regard to the situation identifier.

The corresponding values in the database may originate from a data record that is manually selected. Thus, if a specific situation in which the medical system finds itself is, for example, recognized, a plurality of data records may be offered for selection. Specifically, a plurality of data records may be provided if a situation such as, for example, the heart valve operation has been identified. In this case, the surgeon may select the particular data record that he/she subjectively considers to be the correct one. It is accordingly, for example, possible to use a data record that is always used in a heart valve operation in a renowned hospital.

In one embodiment, the medical system may learn data records with regard to the situation identifier and for the corresponding values in the database to be formed from the data records. Accordingly, for example, a medical procedure can be repeated a number of times and the captured parameter values correspondingly learned. If the medical procedure has been carried out, for example, five times, an optionally averaged data record may be formed from the data records captured in each case and may be stored in the database. This data record is then available for future procedures.

One of the objects is assigned to an object type, where the object of the object type may be used only once in an operating procedure of the medical system, and an inventory control system automatically checks whether a further object of this object type is in stock for a further operating procedure. Not only single-use consumables, such as swabs, but also instruments that always require re-disinfection, such as, for example, scalpels, may accordingly be checked, for example, with regard to absence, in the respective situation. Because they are acquired by the inventory control system, stock levels may be updated. If the respective objects are then out of stock or only small numbers are in stock, a corresponding order may be automatically generated.

In one embodiment, a medical system including an acquisition device for acquiring parameter values that describe a current situation in which the medical system finds itself, and a data processing device (e.g., a data processor) for determining a situation identifier for the current situation from the acquired parameter values is provided. The data processing device is configured for checking the parameter values using corresponding values in a database, from which the corresponding values are obtained based on the determined situation. The medical system also includes a control device (e.g., a controller) for outputting a notification if at least one of the parameter values does not match one of the respective corresponding values or for automatically setting one of the parameter values in accordance with the corresponding value from the database.

The advantages and possible variants explained above in connection with the method according to one or more of the present embodiments are transferable to the medical system. The stated method features are, for example, functional features of corresponding devices such as, for example, the acquisition device, the data processing device, and the output device.

In one specific development, the medical system may include a diagnostic device and, for example, an X-ray machine. The medical system may optionally acquire not only the device itself but also the settings thereof as parameter values. Accordingly, the dose for an X-ray capture may, for example, be acquired as a parameter value. In this case, the dose may be checked against the database and optionally also be automatically modified.

### BRIEF DESCRIPTION OF THE DRAWINGS

FIG. 1 shows a schematic view of a medical system in an exemplary situation; and

FIG. 2 shows a flow chart of one embodiment of a method.

### DETAILED DESCRIPTION

The individual features may be embodied not only in the stated combinations of features, but also alone or in other appropriate technical combinations.

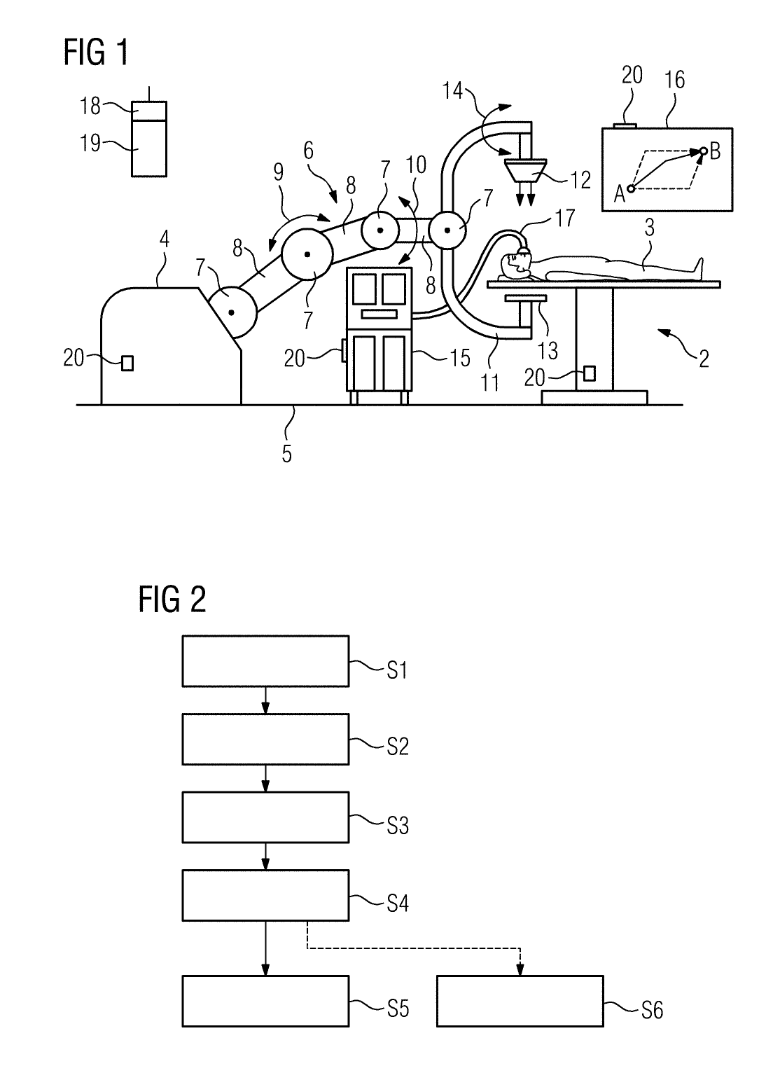

FIG. 1 is a schematic diagram of one embodiment of a medical system with a C-arm X-ray machine 1 and a patient table 2, on which a patient 3 is located. The C-arm X-ray machine 1 has a base 4 that is, for example, fastened rotatably to the floor 5. The C-arm X-ray machine 1 also includes an articulated arm 6, the proximal end of which is swivelably and/or rotatably mounted on the base 4. The articulated arm 6 has, for example, a plurality of articulations 7 and, between each of the articulations 7, a respective arm portion 8. The number of articulations 7 and arm portions 8 may be selected at will. The articulated arm 6 permits movements 9, 10 in various spatial directions. The entire C-arm X-ray machine has, for example, eight axes of movement in order to perform correspondingly complex movements.

A C-arm 11 is arranged at the distal end of the articulated arm 6. An X-ray source 12 is located at one end of the C-arm 11, and a detector 13 at the other end of the C-arm 11. The C-arm 11 may, for example, perform an angulation 14.

In the room (e.g., operating room), an anesthesia device or ventilator 15 is located in the immediate vicinity of the C-arm X-ray machine 1 or patient table 2 supporting the patient 3. A tube 17 leads from the ventilator 15 to the patient 3.

The ventilator 15 and the tube 17 are representative of all the possible objects that jointly describe the entire current situation in which the medical system finds itself. Such objects are also deemed to include other devices or supply systems (e.g., including cables and tubing) and people.

An operator interface 16 (e.g., monitor) is also arranged on the C-arm X-ray machine 1 or in the room (e.g., operating room). This operator interface 16 provides information, for example, about the current medical procedure, about the objects present, about missing objects, or the like.

The medical system also includes an acquisition device 18 with data processing device 19 connected thereto. The acquisition device 18 enables the medical system to acquire the individual objects. The acquisition device 18 may, for example, include a camera for this purpose, and the objects are determined by image processing in the data processing device 19. The acquisition device 18 may, however, also include any desired other sensor systems for acquiring the individual objects 1, 2, 15, 16, etc. Acquisition may proceed in wireless or wired mode. For example, the acquisition device 18 is connected via a data network with one or more objects of the medical system.

Specifically, each object that is to be acquired may, for example, have an RFID tag 20. The acquisition device 18 may thus optionally determine not only the type of the respective object, but also the position thereof as respective parameter values. Under certain circumstances, however, the acquisition device 18 also has additional sensor systems or data interfaces with which further parameter values, such as, for example, temperature, brightness, atmospheric humidity, and the like may be acquired in order to provide a more comprehensive description of the current situation in which the medical system finds itself.

The parameter values acquired by the acquisition device 18, which, for example, relate to the type of the acquired object and the position thereof, are further processed in the data processing device 19. A classification, with which a situation or situation identifier is determined from the acquired parameter values, is, for example, provided. The situation identifier, such as, for example, the term “heart valve operation”, characterizes the situation.

If the system has now sufficiently recognized the situation, the system may retrieve or provide one or more corresponding data records from a database that is, for example, integrated in the data processing device 19. The database may, however, also be provided externally, for example, via a data network. Based on the situation identifier, specific parameters of a C-arm X-ray machine 1 may, for example, then be definitively set. In the case of a specific medical procedure that the system has recognized, an “organ program” of the C-arm X-ray machine 1 may accordingly, for example, be set. In this case, the dose and/or contrast may, for example, be predetermined in order to achieve optimum image quality or a desired appearance.

In another embodiment, the medical system, for example, proposes one or more data records of predetermined doctors. In this manner, the attending doctor may then him/herself decide whether and optionally which of his/her colleagues' programs will be used. Individual steps are also optionally proposed by the medical system in the manner of an assistance system if the corresponding data record has been selected.

Under certain circumstances, the medical system also displays data records for selection that include various guide values. The total X-ray doses for the individual data records may, for example, accordingly be stated. In this manner, a data record in which the medical system manages with a lower X-ray dose than with another data record may, for example, be selected. Based on such guide values, the attending doctor may subjectively decide which parameter settings or parameter values are optimal in a specific situation. These parameter values need not necessarily be parameter values that lead to the best of all possible results. The attending doctor's aim may, for example, be to optimize a parameter other than the X-ray dose.

In a further embodiment, the medical system may be a learning system. In the event of a plurality of medical procedures of the same type, the medical system may accordingly learn which parameter values are used or set in this situation. For example, an ultrasound scanner is, for example, always used during heart valve replacement. If this circumstance is learned by the system and appropriately stored in the database, it is possible to identify when checking the current parameter values that such an ultrasound scanner is missing from the current situation. A suitable notification may accordingly be provided, for example, on the monitor 16 (cf. FIG. 1).

In an alternative application, one or more objects or devices may have settings set in advance. For example, individual devices may accordingly be configured in the same way as configured in the last five procedures. For example, one specific doctor always increases the contrast by two steps in a particular operation. As soon as the system has learned this, the system automatically sets the contrast two steps higher if the corresponding situation (e.g., the doctor) has been recognized. This may apply to overweight patients if the system has learned for such patients that the dose has always been raised, for example, by 10 percent.

It is particularly straightforward for the system to recognize the objects (e.g., people, tools etc.) if the objects are provided with an RFID tag. Other sensor systems may also be used to recognize the current situation. For example, reference may be made to network information for this purpose.

If, for example, the learning system has recognized that three needles and 15 swabs are used in a specific procedure and that another such procedure is scheduled for the following day, this information may be used for an inventory control system. If sufficient stocks of these consumables are no longer available, the consumables may optionally be reordered.

FIG. 2 shows one embodiment of a method for operating a medical system as a block diagram in a plurality of individual acts. A first act S1 involves acquiring parameter values that describe a current situation in which the medical system finds itself. ID tags of objects, positions of objects, settings of devices, and other sensor values are, for example, captured as parameter values. A second act S2 involves determining a situation identifier for the current situation from the acquired parameter values. For example, the situation “heart valve operation” is automatically identified based on the acquired objects.

An optional act S3 involves selecting a data record that is assigned to the determined situation identifier. A database optionally provides a plurality of such data records that may be selected manually, for example, by the attending doctor.

A fourth act S4 then involves checking the parameter values using corresponding values in a database, from which the corresponding values are obtained based on the determined situation identifier. One or more data records including parameter values are thus assigned to the determined situation identifier and compared with the acquired parameter values.

In act S5, a notification may be output if at least one of the parameter values for the current situation does not match one of the respective corresponding values in the database. Thus, if the ultrasound scanner is, for example, missing from the current situation and the corresponding parameter value therefore deviates from the reference value (e.g., corresponding value) in the database, a corresponding indication is displayed, for example, on the monitor 16 (cf. FIG. 1).

Alternatively or in addition to act S5, a sixth act S6 involves automatically setting one of the parameter values in accordance with the corresponding value from the database. Thus, not only is an indication provided but a setting is also actively made on an object or device. The contrast of a C-arm X-ray machine is accordingly automatically raised, for example, by 20 percent.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for operating a medical system, the method comprising:
acquiring parameter values that describe a current situation of the medical system;
determining a situation identifier for the current situation from the acquired parameter values;
checking the parameter values using corresponding values in a database from which the corresponding values are obtained based on the determined situation identifier; and
outputting a notification when at least one the parameter values for the current situation does not match one of the respective corresponding values in the database, or automatically setting one of the parameter values in accordance with the corresponding value from the database.

2. The method of claim 1, wherein the parameter values contain information about which objects are combined to form the medical system.

3. The method of claim 2, wherein the notification is output when, for the determined situation identifier, the corresponding values in the database reveal that at least one object is missing from the objects combined to form the medical system.

4. The method of claim 2, wherein one of the objects is a device, a data network, a tool, or a person.

5. The method of claim 2, wherein each of the objects has an RFID tag, and wherein acquiring the parameter values comprises reading all the RFID tags of the objects of the medical system.

6. The method of claim 1, wherein the corresponding values in the database originate from a data record that is manually selected.

7. The method of claim 1, wherein, with regard to the situation identifier of the medical system, a data record is learned, and the corresponding values in the database are formed from the data record.

8. The method of claim 1, wherein one of the objects is assigned to an object type that is useable only once in an operating procedure of the medical system, and
wherein the method further comprise automatically checking, by an inventory control system, whether a further object of the object type is in stock for a further operating procedure.

9. A medical system comprising:
an acquisition device configured to acquire parameter values that describe a current situation of the medical system;
a data processor configured to;
determine a situation identifier for the current situation from the acquired parameter values; and
check the parameter values using corresponding values in a database from which the corresponding values are obtained based on the determined situation identifier; and
a controller configured to:
output a notification when at least one of the parameter values does not match one of the respective corresponding values; or
automatically set one of the parameter values in accordance with the corresponding value from the database.

10. The medical system of claim 9, further comprising a diagnostic device.

11. The medical system of claim 10, wherein the diagnostic device is an X-ray machine.

12. The medical system of claim 9, wherein the parameter values contain information about which objects are combined to form the medical system.

13. The medical system of claim 12, wherein one of the objects is a device, a data network, a tool, or a person.

14. The medical system of claim 12, wherein each of the objects has an RFID tag, and
wherein the acquisition of the parameter values comprises reading all the RFID tags of the objects of the medical system.

15. The medical system of claim 9, wherein the corresponding values in the database originate from a data record that is manually selected.
